(19) United States
(12) Patent Application Publication
(10) Pub. No.: US 2024/0343667 A1
(43) Pub. Date: Oct. 17, 2024

(54) PROCESS FOR PREPARING ACRYLIC ACID

(71) Applicant: BASF SE, Ludwigshafen am Rhein (DE)

(72) Inventors: Tile GIESHOFF, Ludwigshafen am Rhein (DE); Ulrich HAMMON, Mannheim (DE); Juergen SCHROEDER, Ludwigshafen am Rhein (DE); Peter ZUROWSKI, Ludwigshafen am Rhein (DE); Christian REIN, Ludwigshafen am Rhein (DE); Filip CRAUWELS, Antwerpen (BE)

(21) Appl. No.: 18/292,359

(22) PCT Filed: Jul. 19, 2022
(86) PCT No.: PCT/EP2022/070166

§ 371 Date: Jan. 26, 2024

(30) Foreign Application Priority Data
Jul. 28, 2021 (EP) 21188252.7

(51) Int. Cl. C07C 51/25; C07C 51/43

(52) U.S. Cl. CPC C07C 51/252; C07C 51/43

(57) ABSTRACT

A process for preparing acrylic acid, in which heterogeneously catalyzed gas phase partial oxidation of at least one C₃ precursor of acrylic acid with molecular oxygen over catalysts in the solid state of matter at elevated temperature affords a product gas mixture comprising acrylic acid, water vapor and secondary components, then the product gas mixture is directed into a condensation column equipped with separating internals, the product gas mixture is allowed to ascend into itself within the condensation column and undergoes fractional condensation, separating the product gas mixture into a bottoms liquid comprising conversion products and secondary components that are higher-boiling than acrylic acid, a crude acrylic acid comprising water and secondary components that have been depleted overall as target product, an acid water still comprising acrylic acid and secondary components, and a residual gas mixture comprising secondary components that are lower-boiling than water, the target product is conducted out of the condensation column via a side draw and the side draw is above the feed point of the product gas mixture into the condensation column, wherein the parts of the condensation column that are in contact with product are made of stainless steel, at least one of the streams of matter fed to the condensation column comprises a source for halide ions, and halide ions are removed in the region of the separating internals of the condensation column above the side draw.

A process for preparing acrylic acid, in which heterogeneously catalyzed gas phase partial oxidation of at least one C₃ precursor of acrylic acid with molecular oxygen over catalysts in the solid state of matter at elevated temperature affords a product gas mixture comprising acrylic acid, water vapor and secondary components, then the product gas mixture is directed into a condensation column equipped with separating internals, the product gas mixture is allowed to ascend into itself within the condensation column and undergoes fractional condensation, separating the product gas mixture into a bottoms liquid comprising conversion products and secondary components that are higher-boiling than acrylic acid, a crude acrylic acid comprising water and secondary components that have been depleted overall as target product, an acid water still comprising acrylic acid and secondary components, and a residual gas mixture comprising secondary components that are lower-boiling than water, the target product is conducted out of the condensation column via a side draw and the side draw is above the feed point of the product gas mixture into the condensation column, wherein the parts of the condensation column that are in contact with product are made of stainless steel, at least one of the streams of matter fed to the condensation column comprises a source for halide ions, and halide ions are removed in the region of the separating internals of the condensation column above the side draw.

Acrylic acid is an important intermediate which finds use, for example, in the production of polymer dispersions (possibly also in the form of their esters with alkanols) and of wa-ter-superabsorbing polymers.

Acrylic acid is obtainable, inter alia, by heterogeneously catalyzed gas phase partial oxidation of C₃ precursors (C₃ precursor compounds) of acrylic acid (this term is intended to encompass, more particularly, those chemical compounds which are obtainable in a formal sense by reduction of acrylic acid; known C₃ precursors of acrylic acid are, for example propane, propene, acrolein, propionaldehyde and propionic acid; however, the term is also intended to encompass precursor compounds of the aforementioned compounds, for example glycerol (proceeding from glycerol, acrylic acid can be obtained, for example, by heterogeneous catalyzed oxidative dehydration in the gas phase; cf., for example, EP 1 710 227 A1, WO 06/114506 and WO 06/092272) with molecular oxygen over solid-state catalysts at elevated temperature.

This involves diluting the output gases mentioned, generally with inert gases, for example nitrogen, CO2, saturated hydrocarbons and/or water vapor, directing them in a mixture with molecular oxygen at elevated temperatures and optionally elevated pressure over (for example transition metal) mixed oxide catalysts, and converting them oxida-tively to a product gas mixture comprising acrylic acid, water and unwanted by-products, for example furfurals, benzaldehyde, acetone, formaldehyde and maleic anhydride etc., from which the acrylic acid has to be separated (the by-products and the inert diluent gases other than water vapor shall be referred to collectively in this document by the term “secondary components”; in addition, this term shall include the polymerization inhibitors that are typically added in the acrylic acid removal methods).

Documents DE 199 24 533 A1, DE 199 24 532 A1, WO 01/77056, DE 101 56 016 A1, DE 102 43 625 A1, DE 102 23 058 A1, DE 102 35 847 A1, WO 2004/035514, WO 00/53560, DE 103 32 758 A1 and EP 2 114 852 A1 disclose processes for preparing acrylic acid as described at the outset, in which a basic removal of a crude acrylic acid by fractional condensation of the product gas mixture from the heterogeneously catalyzed gas phase partial oxidation is undertaken. The term “crude acrylic acid” expresses the fact that acrylic acid withdrawn via the first side draw is not a pure product but a mixture comprising, as well as acrylic acid (generally ≥50% or ≥60% by weight, usually >70% or ≥80% by weight, in many cases≥90% by weight and frequently ≥95% by weight or more of the total weight), also water and secondary components, for example lower aldehydes (e.g. furfurals, acrolein, benzaldehyde), lower carboxylic acids (e.g. acetic acid, propionic acid, formic acid) etc. In each case, the total content of water and second components, based on the acrylic acid content, in the crude acrylic acid is lower than in the product gas mixture from the gas phase partial oxidation, and therefore it is also said that the crude acrylic acid comprises these constituents depleted overall (individual constituents, by contrast, may be comparatively enriched in the crude acrylic acid).

In some cases, the purity of the crude acrylic acid thus removed is already sufficient for the contemplated end use of the acrylic acid (for example for the purpose of esterification thereof, or for the purpose of forming polymers obtainable by free-radical polymerization). In many cases, however, the crude acrylic acid removed will be subjected to a further thermal separation process in order to obtain, from the crude acrylic acid, a purer acrylic acid (one having a higher acrylic acid content in % by weight by comparison with the crude acrylic acid) that has the level of purity required for the respective end use.

Thermal separation processes are understood to mean those in which a physically at least biphasic system is created with supply or withdrawal of (generally thermal) energy, with transfer of heat and mass as a result of the gradients of temperature and molarity that exist between the phases, which ultimately results in the desired separation, and extraction.

Frequently, thermal separation processes are conducted in separation columns comprising separating internals in which the aforementioned at least two physical phases are generally conducted in countercurrent to one another. In many cases, one of the two physical phases is gaseous (which is generally run as the ascending phase in a separation column) and the other is liquid (which is generally run as the descending phase in a separation column). In principle, the at least two physical phases may also be liquid (for example in the case of an extraction) or solid and liquid (for example in the case of a crystallization), or solid and gaseous (for example in the case of an adsorp-tion).

Examples of cases of thermal separation processes in which one of the at least two physical phases is in liquid form and one in gaseous form, and hence a natural element of the term “thermal separation process” used in this document, are rectification (an ascending vapor phase is run in countercurrent to a descending liquid phase in the separation column) and desorption (the reverse process of absorption; the gas dissolved in a liquid phase is extracted from the liquid phase by lowering the pressure over the liquid phase, by increasing the temperature of the liquid phase and/or by passing a gas phase through the liquid phase; if the conduction of a gas phase through the liquid phase is in-volved, the desorption is also referred to as stripping). But absorption (in general, a gas ascending within a separation column is run in countercurrent to at least one absorbent descending in liquid form in the separation column) and fractional condensation of a gas mixture (gas/liquid phase example) are also embraced by the term “thermal separation process”. A particularly favorable thermal separation process for further purification of crude acrylic acid is crystallizative further purification (crystallization).

In the fractional crystallization of the product gas mixture from heterogeneously catalyzed gas phase partial oxidation, corrosion is occasionally and unexpectedly found in the condensation column used.

The problem addressed was that of preventing this unexpected corrosion.

The problem is solved by a process for preparing acrylic acid, in which heterogeneously catalyzed gas phase partial oxidation of at least one C₃ precursor of acrylic acid with molecular oxygen over catalysts in the solid state of matter at elevated temperature affords a product gas mixture comprising acrylic acid, water vapor and secondary components, then the product gas mixture is directed into a condensation column equipped with separating internals, the product gas mixture is allowed to ascend into itself within the condensation column and undergoes fractional condensation, separating the product gas mixture into a bottoms liquid comprising conversion products and secondary components that are higher-boiling than acrylic acid, a crude acrylic acid comprising wa-ter and secondary components that have been depleted overall as target product, an acid water still comprising acrylic acid and secondary components, and a residual gas mixture comprising secondary components that are lower-boiling than water, the target product is conducted out of the condensation column via a side draw and the side draw is above the feed point of the product gas mixture into the condensation column, wherein the parts of the condensation column that are in contact with product are made of stainless steel, at least one of the streams of matter fed to the condensation column comprises a source for halide ions, and halide ions are removed in the region of the separating internals of the condensation column above the side draw.

The present invention is based on the finding that halide ions can accumulate in the condensation column above the side draw. These halide ions are the cause of the unexpected corrosion. Where the concentration of the halide ions is at its highest, the great-est corrosion is also found. The corrosion can be avoided if the halide ions are removed in a controlled manner.

The C₃ precursor of acrylic acid is preferably propene and/or acrolein.

The stream of matter comprising halide ions may, for example, be water, propene, sodium hydroxide solution, hydroquinone, hydroquinone monomethyl ether, diethyl phthalate and/or phenothiazine. The halide ions may be present as an impurity in these or other streams of matter fed to the process.

Typically, fluoride ions and chloride ions are the halide ions found.

The condensation column preferably comprises dual-flow trays and crossflow trays as separating internals.

The parts of the condensation column that are in contact with product are made from stainless steel. Stainless steels in the context of this invention are steels having at least 10.5% by weight of chromium.

The preferred stainless steels preferably comprise 16.0% to 21.0% by weight, more preferably 17.0% to 20.5% by weight, most preferably 18.0% to 20.0% by weight, of chromium, and more preferably additionally preferably 8.0% to 26.0% by weight, more preferably 10.0% to 25.0% by weight, most preferably 12.0% to 24.0% by weight, of nickel, and/or additionally preferably 2.0% to 5.0% by weight, more preferably 2.5% to 4.5% by weight, most preferably 3.0% to 4.0% by weight, of molybdenum.

In addition, the stainless steels may advantageously preferably comprise 1.2% to 2.0% by weight, more preferably 1.3% to 1.9% by weight, most preferably 1.4% to 1.8% by weight, of copper.

In a preferred embodiment of the present invention, a liquid F is withdrawn from the condensation column in the region of the separating internals of the condensation column above the side draw. The amount of liquid F withdrawn is preferably from 0.0001% to 0.5% by weight, more preferably from 0.001% to 0.4% by weight, most preferably from 0.01% to 0.3% by weight, based in each case on the crude acrylic acid withdrawn in the side draw.

The halide ions can be removed from the liquid F withdrawn, for example chloride ions by means of a basic ion exchanger. Subsequently, the liquid F that has been freed of halide ions can be recycled back into the condensation column.

It is also possible to remove halide ions directly in the condensation column, for example fluoride ions by reaction with glass that has been introduced into the condensation column for that purpose.

Alternatively, the liquid F withdrawn can also be combined with the bottoms liquid discharged from the condensation column and worked up together therewith.

Alternatively, the liquid F withdrawn can also be combined with the acid water discharged from the condensation column and worked up together therewith.

The amount of halide ions which is removed from the condensation column should be chosen such that the halide ion content in the streams of matter from the condensation column is preferably less than 0.005% by weight, more preferably less than 0.002% by weight, most preferably less than 0.001% by weight, based in each case on the stream of matter. This means that the concentrations should be lower than those given above in every part of the condensation column.

The preparation of acrylic acid is described hereinafter:

Typically, the acrylic acid-comprising product gas mixture from a heterogeneously catalyzed gas phase partial oxidation of C₃ precursors of acrylic acid with molecular oxygen over catalysts in the solid state may have, for example, the following contents (especially when the C₃ precursor used is propene):
1% to 30% by weight of acrylic acid,
0.05% to 10% by weight of molecular oxygen,
1% to 30% by weight of water,
>0% to 5% by weight of acetic acid,
>0% to 3% by weight of propionic acid,
>0% to 1% by weight of maleic acid and/or maleic anhydride,
0% to 2% by weightof acrolein,
0% to 1% by weightof formaldehyde,
>0% to 1% by weight of furfurals,
>0% to 0.5% by weight of benzaldehyde,
0% to 1% by weightof propene, and as the remaining amount essentially inert gases, for example nitrogen, carbon monoxide, carbon diox-ide, methane and/or propane.

Typically, the product gas mixture, based on acrylic acid present, comprises ≥0.005 mol %, frequently ≥0.03 mol %, of furfurals. In general, however, the furfural content on this basis is ≤3 mol %.

The gas phase partial oxidation itself can be conducted as described in the prior art. Proceeding from propene, the gas phase partial oxidation can be conducted, for example, in two successive oxidation stages, as described in EP 0 700 714 A1 and in EP 0 700 893 A1. It is of course also possible to employ the gas phase partial oxidations cited in DE 197 40 253 A1 and in DE 197 40 252 A1.

For the purposes of a small amount of secondary components formed, the propene gas phase partial oxidation is preferably conducted as described in DE 101 48 566 A1. The propene source used for this purpose may be polymer grade propene or chemical grade propene according to DE 102 32 748 A1. If the C₃ precursor used is propane, the par-tial oxidation can be conducted as described in DE 102 45 585 A1.

In principle, the gas phase partial oxidation can also be conducted as described in documents US 2006/0161019, WO 2006/092410, WO 2006/002703, WO 2006/002713, WO 2005/113127, DE 10 2004 021 763 A1, EP 1 611 076 A1, WO 2005/108342, EP 1 656 335 A1, EP 1 682 478 A1, EP 1 682 477 A1, DE 10 2006 054 214 A1, DE 10 2006 024 901 A1, EP 1 611 080 A2, EP 1 734 030 A1, DE 10 2006 000 996 A1, DE 10 2005 062 026 A1, DE 10 2005 062 010 A1, WO 2007/060036, WO 2007/051750 and WO 2007/042457.

Frequently, the temperature of the product gas mixture leading the gas phase partial oxidation is 150 to 350° C., in many cases 200 to 300° C., sometimes up to 500° C.

Appropriately for application purposes, the hot product gas mixture is then cooled down in a quench apparatus 1 by direct cooling, generally to a temperature of 100 to 180° C., before it is routed, advantageously for application purposes together with the quench liquid 1 used, for the purpose of fractional condensation, preferably into the lower section (preferably the lowermost section, for example the bottom space) of a condensation column comprising separating internals.

Useful condensation column internals in principle include all standard internals, especially trays, structure packings and/or random packings. Of the trays, preference is given to bubble-cap trays, sieve trays, valve trays and/or dual-flow trays. Typically, in a tray column, the total number of separation trays is 20 to 100, frequently 20 to 80 and preferably 50 to 80.

Preferably in accordance with the invention, the condensation column is one comprising, as separating internals, from the bottom upward, firstly dual-flow trays and subsequently hydraulically sealed crossflow trays (e.g. Thormann trays), as recommended by DE 102 43 625 A1, DE 199 24 532 A1 and DE 102 43 625 A1. The number of dual-flow trays may be 5 to 60, frequently 25 to 45, and the number of hydraulically sealed crossflow trays may likewise be 5 to 60, frequently 30 to 50. For the region of acid water formation (acrylic acid content of the reflux liquid viewed from the bottom upward generally ≤15% by weight, or in some cases≤10% by weight), useful separating internals are preferably valve trays, as described by DE 199 24 532 A1 and DE 102 43 625 A1. In principle, it would also be possible to use other standard separating internals (the individual regions within the condensation column may of course be configured in an en-tirely equivalent manner (rather than one on top of another in a column) as a series con-nection of correspondingly smaller columns).

The quench apparatus 1 used may be any of the apparatuses known from this purpose in the prior art (for example spray scrubbers, Venturi scrubbers, bubble columns or other apparatuses with surfaces over which liquid trickles), preference being given to using Venturi scrubbers or spray coolers.

For indirect cooling or heating of the quench liquid 1, especially on startup, it is preferably but not necessarily directed through a heat transferer or heat exchanger. All standard heat transferers or heat exchangers are suitable in this regard. Preference is given to shell and tube heat exchangers, plate heat exchangers and air coolers. Suitable cooling media are air in the case of the corresponding air cooler, and cooling liquids, especially water, in the case of the other cooling apparatus.

The quench liquid 1 used may, for example, be bottoms liquid withdrawn from the bottom of the condensation column (optionally combined with condensate conducted out of the quench circuit 0), or high boiler fraction via a side draw close to the bottom, or a mixture of such bottoms liquid and high boiler fraction (especially when the bottom space and the lowermost theoretical plate (lowermost separating internal) are separated by a chimney tray). It may be the case that only the proportion of the quench liquid 1 which is withdrawn from the bottom of the condensation column is directed through the abovementioned heat exchanger. The temperature of the quench liquid 1 on entry into the quench apparatus 1 is generally appropriately 90° C. to 120° C.

The site of introduction for the product gas mixture from the gas phase partial oxidation that has been quenched (or cooled in some other way or not cooled) (according to the invention, as described, preferably in a mixture with quench liquid 1 used for direct cooling) into the condensation column is advantageously in the bottom space of that column, which advantageously comprises a centrifugal droplet separator in integrated form and is generally separated from the lowermost separating internal by a first chimney tray (appropriately from application point of view, in that case, high boiler fraction is con-stantly directed into the bottom of the condensation column via a connecting conduit or via an overflow). In an illustrative and preferred execution variant (which is described exclusively hereinafter without restriction of general implementability), this is the first dual-flow tray of a first series of dual-flow trays that are appropriately arranged equidis-tantly. The chimney tray functions simultaneously as collecting tray, from which condensate (high boiler fraction) is withdrawn continuously and run as part of the quench liquid 1 into the quench apparatus 1 or into the bottom space. The first series of dual-flow trays is concluded by a second chimney tray (collecting tray). From this second chimney tray, in the first side draw, crude acrylic acid is withdrawn continuously as medium boiler fraction, preferably having a purity of ≥90% by weight or ≥95% by weight.

Appropriately, this crude acrylic acid will be sent to further distillative (rectificative) and/or crystallizative further purification stages, and at least a portion of the bottoms liquids and/or mother liquors obtained in this distillation (rectification) and/or crystallization will be recycled into the condensation column below the first side draw, but above the first collecting tray. This recycling is preferably effected in a thermally integrated manner. In other words, cold mother liquor to be recycled is run through one or more series-connected indirect heat exchangers (e.g. spiral heat exchangers) in order to cool down therein the crude acrylic acid that has been withdrawn from the condensation column and is to be subjected to further purification by crystallization on the opposite side in the heat exchanger. At the same time, this heats up the mother liquor. Preferably, for this purpose, two series-connected plate heat exchangers are used.

Appropriately, the crude acrylic acid withdrawn (as medium boiler fraction) will be sent to a crystallization for the purpose of further purification. The crystallization method to be used is not subject to any restriction in principle. The crystallization can be conducted continuously or batchwise, in one or more stages, up to any degree of purity.

If required, the crude acrylic acid to be purified by crystallization can advantageously be admixed with water (in general, it then comprises, based on the amount of acrylic acid present, up to 20% by weight or up to 10% by weight, usually up to 5% by weight, of water). In the case of elevated aldehyde or other secondary component contents, there may be no need to add water since the aldehydes in this case are capable of assuming the function of water. Very particularly advantageously in accordance with the invention, the water is added in the form of acid water. This leads to an increase in the yield of glacial acrylic acid.

It is surprising that, even in the case of prior addition of acid water to the crude acrylic acid (this measure likewise results in an increase in acrylic acid yield), an acrylic acid (of purity≥98% by weight) which meets the highest demands and is of esterification grade (for example for the preparation of n-butyl acrylate, 2-ethylhexyl acrylate, methyl acrylate and ethyl acrylate) can already be achieved by a single crystallization stage.

Appropriately, this crystallization stage is executed as a suspension crystallization, as described in column 10 of DE 199 24 532 A1 or in example 1 of DE 102 23 058 A1 (for example in a cooling disk crystallizer as described in WO 2006/111565). The acrylic acid crystals formed in suspension crystallization have a cubic to cuboidal appearance. The ratio of length (L) to thickness (D) is typically in the range of L:D=1:1 to L:D=6:1, preferably in the range from 1:1 to 4:1 and more preferably in the range from 1.5:1 to 3.5:1. The thickness D of the crystals is typically in the range from 20 to 600 μm, often 50 to 300 μm. The length L of the crystals is typically in the range from 50 to 1500 μm, often 200 to 800 μm. The suspension crystals can be separated from the remaining mother liquor in the case of esterification grade acrylic acid in a centrifuge (for example a 2- or 3-stage pusher centrifuge), in which case the crystals removed are advantageously washed on the centrifuge by means of molten pure crystals. If the suspension crystals are separated from the remaining mother liquor by means of a scrubbing column, for example a melt scrubbing column (for example one according to WO 01/77056, or DE 101 56 016 A1, or DE 102 23 058 A1, or as described in WO 2006/111565, WO 04/35514, WO 03/41833, WO 02/09839, WO 03/41832, DE 100 36 881 A1, WO 02/55469 and WO 03/78378), it is possible by means of a single crystallization stage even to achieve superabsorbent grade acrylic acid (purity≥99.7% by weight or ≥99.9% by weight), i.e. acrylic acid suitable for production of water-super ab-sorbing or other polyacrylates. In this case, in an appropriate manner, the entire amount of mother liquor separated off is recycled into the condensation column.

But the crystallization can also be performed as a fractional falling film crystallization, as recommended by EP 0 616 998 A1. This may comprise, for example, two, three or more (e.g. 2 to 4) purification stages (falling film crystallizers that are suitable in this regard may comprise, for example, 1000 to 1400 crystallization tubes of length 10 to 15 m and external diameter 50 to 100 mm). The mother liquor separated off at a higher purification stage may be recycled into one of the preceding purification stages. The mother liquor separated off at the first purification stage is advantageously recycled fully into the condensation column. As an alternative to recycling into one of the preceding purification stages, the mother liquors from the individual purification stages may also be recycled into the condensation column in their entirety. The pure product from the penulti-mate purification stage may be sent fully or only partly to the last purification stage. If it is sent only partly, the remaining residual amount will generally be blended with the pure product from the last purification stage to give the end product which is then suitable for use.

Appropriately in accordance with the invention, a portion of the crude acrylic acid withdrawn via the first side draw will be fed to the dual-flow tray below the collecting tray belonging thereto. This tray will generally also be supplied with any mother liquor to be recycled into the condensation column. Before the supply, the mother liquor will generally, as already described, be heated by thermal integration to a temperature corresponding roughly to the withdrawal temperature of the crude acrylic acid.

Another portion of the crude acrylic acid withdrawn by the first side draw will advantageously be heated by 10 to 15° C. by indirect heat exchange and recycled into the condensation column above the feed point, preferably immediately below the first subse-quent dual-flow tray. This measure has a favorable effect on the acetic acid content of the crude acrylic acid withdrawn.

Above the second collecting tray there is firstly a second series of appropriately equidistant dual-flow trays, which are then followed by hydraulically sealed crossflow mass transfer trays (e.g. Thormann trays or modified Thormann trays according to DE 102 43 625 A1) that are appropriately likewise in an equidistant arrangement. The uppermost dual-flow tray may be modified as a distributor tray. In other words, it has, for example, overflow grooves with a zigzag overflow.

The first of the Thormann trays from the bottom, appropriately for application purposes, is one in which the liquid running off the tray runs off via six downcomers in the form of pipes. These pipes are hydraulically sealed with respect to the gas space of the dual-flow tray beneath. The weir heights of the six downcomers, appropriately for application purposes, decrease in flow direction of the crossflow tray. Advantageously, the hydraulic seal has emptying orifices with a deflector plate. The downcomers are preferably dis-tributed uniformly in the second half, more preferably in the last third, of the tray cross section (opposite the feed to the trays).

The hydraulic seal is made in a cup with an oblique overflow weir (45°).

The crossflow mass transfer trays are concluded by a third chimney tray (collecting tray).

Above the third collecting tray are valve trays, preferably dual-flow valve trays. The principle of valve trays and of valve trays usable in accordance with the invention can be found, for example, in Technische Fortschrittsberichte [Industrial Progress Reports], vol-ume 61, Grundlagen der Dimensionierung von Kolonnenboden [Fundamentals of the Dimensioning of Tray Columns], pages 96 to 138. They are essentially characterized in that they provide a passage opening corresponding to the respective load to the steam flowing through over a wide load range. Preferably in accordance with the invention, ballast trays are used. In other words, there are cages with openings closed by weights in the openings of the tray. Particular preference is given in accordance with the invention to VV12 valves from Stahl, Viernheim, Germany. Constituents that condense in the valve tray space are essentially water and constituents less volatile than water. The condensate obtained is acid water.

The acid water is withdrawn continuously from the first collecting tray in the second side draw. A portion of the acid water withdrawn is recycled into the condensation column at the uppermost of the crossflow mass transfer trays. Another portion of the acid water withdrawn is cooled by indirect heat exchange, appropriately split, and likewise recycled into the condensation column. A cooled portion is recycled here to the uppermost valve tray (at a temperature of 15 to 25° C., preferably 20 to 25° C.), and the other cooled portion to a valve tray disposed roughly in the middle between the third collecting tray and the uppermost valve tray (at a temperature of 20 to 35° C., preferably 25 to 30° C.). The amount of acrylic acid present can be separated in accordance with the invention from the residual amount of acid water withdrawn.

A portion of the cooling (which can be undertaken by means of one or more series-connected indirect heat exchangers) is brought about in that the appropriate portion of acid water is guided through the evaporator of the C₃ precursor (e.g. the propene evaporator) in order to convert C₃ precursors stored in liquid form, e.g. propene, to the gas phase for the heterogeneously catalyzed gas phase oxidation.

The constituents that are more volatile than water are drawn off in gaseous form at the top of the condensation column as residual gas (residual gas mixture), and are normally recycled into the gas phase partial oxidation as diluent gas (cycle gas). In order to avoid condensation in the cycle gas compressor, the residual gas mixture is superheated beforehand by indirect heat exchange. The portion of the residual gas mixture that has not been circulated is normally sent to incineration. A portion of the (preferably compressed) residual gas mixture, as already described, is advantageously used as stripping gas for separation of acrylic acid from the extract and from the bottoms liquid from the condensation column. Advantageously, the gas phase partial oxidation is conducted with an ex-cess of molecular oxygen, such that the residual gas mixture and hence the first and second stripping gas comprise molecular oxygen if residual gas mixture is used as such stripping gas.

For the purpose of inhibiting polymerization, the uppermost of the hydraulically sealed crossflow mass transfer trays is supplied with a solution of hydroquinone monomethyl ether (=MEHQ) in acrylic acid or (preferably in accordance with the invention) an MEHQ melt and (in both cases) optionally additionally a solution of phenothiazine in acrylic acid. The acrylic acid used here is preferably a pure acrylic acid as produced in the further purification of the crude acrylic acid withdrawn. For example, it is possible to use the glacial acrylic acid (pure product) produced in crystallizative further purification. The solution is appropriately also used for pure product stabilization.

In addition, roughly in the middle of the column section comprising the hydraulically sealed crossflow mass transfer trays, it is possible to feed in a solution of phenothiazine (=PTZ) in pure product.

In principle, acid water formation can also be implemented, for example, downstream of a first condensation column (cf. DE 102 35 847 A1). In this case, essentially water will be condensed out of the low boiler gas stream which then leaves the first condensation column at the top, appropriately by direct cooling in a downstream space (second column) which is free of internals or comprises internals, by means of a quench liquid 2. The condensate obtained is again the acid water. A portion of the acid water will then advisably be recycled into the first condensation column in order to increase the separation performance at the top thereof. A further portion of the acid water will be cooled indirectly in an external heat exchanger and be used as the quench liquid 2, and the acrylic acid can in turn be extracted in accordance with the invention from the residual amount of acid water. Constituents of the low boiler stream that are more volatile than water in turn form residual gas, which is normally at least partly recycled into the gas phase partial oxidation as cycle gas or used as stripping gas.

Appropriately, the dual-flow trays in the preferred variant of the process of the invention extend within the condensation column up to about the cross section of the condensation column from which the acrylic acid contents of the reflux liquid towards of the column are ≤90% by weight, based on the weight of the reflex liquid.

The number of dual-flow trays, as already stated, for the described preferred variant of the fractional condensation is generally 25 to 45. The opening ratio thereof is appropriately 12% to 25%. As passages, the dual-flow trays preferably have circular holes having a uniform circle diameter. The latter is appropriately 10 to 20 mm. If required, the hole diameters in the condensation column may narrow or increase from the top downward and/or the number of holes can decrease or increase (for example, the hole diameter may be a uniform 14 mm, and the opening ratio from the top downward may increase from 17.4% to 18.3%). However, the number of holes may also be constant over all dual-flow trays. In addition, the circular holes are arranged uniformly, preferably in strict triangular pitch, over the individual dual-flow trays (cf. DE 102 30 219 A1).

Moreover, the punch burr in the passages punched out in the dual-flow trays preferably points downward in the condensation column (this reduces unwanted polymer formation).

It is advisable in accordance with the invention when the number of dual-flow trays used in the condensation column corresponds to about 10 to 15 theoretical plates.

The number of hydraulically sealed crossflow mass transfer trays that follows the dual-flow trays in the condensation column preferred in accordance with the invention, as already mentioned, will generally be 30 to 50. The opening ratio will appropriately be 5% to 25%, preferably 10% to 20% (the opening ratio quite generally reflects the percent-age of the cross sections of passages in the total cross section; it is quite generally appropriately within the aforementioned range in the crossflow mass transfer trays that are to be used with preference).

Single-flow crossflow mass transfer trays are used with preference in accordance with the invention.

In general, the number of hydraulically sealed crossflow trays for the preferred variant of the fractional product gas mixture condensation is such that it corresponds to about 10 to 30, frequently 25, theoretical plates.

Both the hydraulically sealed crossflow trays and any valve trays that are also used have at least one downcomer. They may have either a single-flow or multiflow configuration, for example a dual-flow configuration. Even in the case of a single-flow configuration, they may have more than one downcomer. In general, the downcomers of the valve trays are also hydraulically sealed.

The inhibition of polymerization in the current system 1 for the product gas mixture from the partial gas phase oxidation can be accomplished either by means of polymerization inhibitors present via bottoms liquid used for quenching (from the condensation column) or by means of polymerization inhibitors present via high boiler fraction used for quenching (from the condensation column).

Another reason why the process of the invention is advantageous is that it enables an elevated yield of crude acrylic acid with essentially the same purity. All statements made in this document are especially applicable to a product gas mixture that has been obtained by (preferably two-stage) heterogeneous partial oxidation of propene to acrylic acid. The above-described preferred execution variant of the process of the invention does not restrict general implementability in any way.

Finally, it should be emphasized that of the first ripping gas and the second stripping gas advantageously comprise molecular oxygen.

### EXAMPLES

### Example 1 (Comparative Example)

The procedure was as in example 1 of EP 2 114 852 A1. Chloride introduced accumu-lated in the region of the Thormann trays. The chloride content was up to 115 ppm. The Thormann trays were made of stainless steel (1.4571 material according to DIN EN 10088:16.5% to 18.5% by weight of chromium, 10.5% to 13.5% by weight of nickel, 2.0% to 2.5% by weight of molybdenum, up to 0.7% by weight of titanium). Corrosion was observed on Thormann trays 5 to 7 (counted from the lowermost Thormann tray).

### Example 2

The procedure was as in example 1. 80 to 205 kg/h of liquid was drawn off from Thormann tray 6 (counted from the lowermost Thormann tray). The amount of liquid is controlled such that the chloride content remains below 10 ppm. The liquid discharged can be recycled into the acid water extraction or the second stripping column.

1.-15. (canceled)

16. A process for preparing acrylic acid, in which heterogeneously catalyzed gas phase partial oxidation of at least one C₃ precursor of acrylic acid with molecular oxygen over catalysts in the solid state of matter at elevated temperature affords a product gas mixture comprising acrylic acid, water vapor and secondary components, then the product gas mixture is directed into a condensation column equipped with separating internals, the product gas mixture is allowed to ascend into itself within the condensation column and undergoes fractional condensation, separating the product gas mixture into a bottoms liquid comprising conversion products and secondary components that are higher-boiling than acrylic acid, a crude acrylic acid comprising water and secondary components that have been depleted overall as target product, an acid water still comprising acrylic acid and secondary components, and a residual gas mixture comprising secondary components that are lower-boiling than water, the target product is conducted out of the condensation column via a side draw and the side draw is above the feed point of the product gas mixture into the condensation column, wherein the parts of the condensation column that are in contact with product are made of stainless steel, at least one of the streams of matter fed to the condensation column comprises a source for halide ions, and halide ions are removed in the region of the separating internals of the condensation column above the side draw.

17. The process according to claim 16, wherein the halide ions are fluoride ions and/or chloride ions.

18. The process according to claim 16, wherein the C₃ precursor of acrylic acid is propene and/or acrolein.

19. The process according to claim 16, wherein the stream of matter comprising a source for halide ions is water, propene, sodium hydroxide solution, hydroquinone, hydroquinone monomethyl ether, diethyl phthalate and/or phenothiazine.

20. The process according to claim 16, wherein dual-flow trays and crossflow trays are used as separating internals in the condensation column.

21. The process according to claim 16, wherein the parts of the condensation column that are in contact with product are made from stainless steel having 16% to 21% by weight of chromium and 8% to 26% by weight of nickel.

22. The process according to claim 21, wherein the parts of the condensation column that are in contact with product are made from stainless steel additionally having 2% to 5% by weight of molybdenum.

23. The process according to claim 22, wherein the parts of the condensation column that are in contact with product are made from stainless steel additionally having 1.2% to 2.0% by weight of copper.

24. The process according to claim 16, wherein a liquid F is withdrawn from the condensation column in the region of the separating internals of the condensation column above the side draw.

25. The process according to claim 24, wherein from 0.0001% to 0.5% by weight of liquid F is withdrawn, based on the crude acrylic acid withdrawn in the side draw.

26. The process according to claim 24, wherein halide ions are removed from the liquid F, and then the liquid F is recycled into the condensation column.

27. The process according to claim 26, wherein the halide ions are removed from the liquid F by means of a basic ion exchanger.

28. The process according to claim 24, wherein the liquid F is combined with the bottoms liquid discharged from the condensation column.

29. The process according to claim 24, wherein the liquid F is combined with the acid water discharged from the condensation column.

30. The process according to claim 16, wherein the halide content in the streams of matter from the condensation column is less than 0.002% by weight, based on the stream of matter.
